(19) United States
(12) Patent Application Publication
(10) Pub. No.: US 2023/0219815 A1
(43) Pub. Date: Jul. 13, 2023

(54) SYSTEM NETWORK AND METHOD FOR OPERATING A SYSTEM NETWORK OF THIS TYPE FOR PRODUCING HIGHER ALCOHOLS

(71) Applicants: thyssenkrupp Industrial Solutions AG, Essen (DE); thyssenkrupp AG, Essen (DE)

(72) Inventors: Nils TENHUMBERG, Dortmund (DE); Stefan GEHRMANN, Dortmund (DE)

(73) Assignees: thyssenkrupp Industrial Solutions AG, Essen (DE); thyssenkrupp AG, Essen (DE)

(21) Appl. No.: 18/014,510

(22) PCT Filed: Jun. 22, 2021
(86) PCT No.: PCT/EP2021/066958

§ 371 Date: Jan. 5, 2023

(30) Foreign Application Priority Data
Jul. 7, 2020 (DE) 10 2020 208 458.0

(51) Int. Cl. C01B 3/32; C21B 7/00; C21C 5/28

(52) U.S. Cl. CPC C01B 3/32; C21B 7/002; C21C 5/285; C01B 2203/042; C21B 2100/282; C21B 2100/22

(57) ABSTRACT

A plant complex may include a unit that produces CO₂-containing gases, a gas conducting system for CO₂-containing gases, a gas/liquid separation system, a reformer that is connected to the gas conducting system and where the CO₂-containing gas reacts with H₂ and/or hydrocarbons to give a CO— and H₂-containing synthesis gas mixture. The reformer is connected to a reactor for producing higher alcohols in which the synthesis gas mixture reacts with H₂ to give a gas/liquid mixture containing higher alcohols. For separating off the alcohols of the gas/liquid mixture, the gas/liquid separation system is connected to the reactor for producing higher alcohols.

The invention relates to a plant complex comprising a unit that produces CO₂-containing gases, a gas conducting system for CO₂-containing gases and a gas/liquid separation system. The invention further relates to a process for producing higher alcohols from CO₂-containing gases with a plant complex comprising a unit that produces CO₂-containing gases, a gas conducting system for CO₂-containing gases, a reformer, a reactor for producing higher alcohols and a gas/liquid separation system.

Steel, pig iron and coke production produce large amounts of smelter gases, in particular blast furnace gas, converter gas and coke oven gas, where some of these gases can be recycled but a not insignificant proportion thereof is converted into electricity. However, this high degree of conversion to electricity is accompanied by a high undesired CO₂ emission. This problem does not exist only in the sector of steel, pig iron and coke production, but also applies to numerous other industrial applications using units that produce CO₂-containing gases.

In general, there are various approaches for using the CO₂ for the synthesis of chemical products, in particular alcohols and hydrocarbons.

The 1980s saw the development of what is known as the Snamprogetti, Enichem and Haldor Topsoe (SEHT) process. This is targeted at generating, proceeding from the starting materials natural gas and coal, an essentially CO₂-free synthesis gas from which methanol and higher alcohols can be produced.

Synthesis gases, which primarily contain carbon monoxide and hydrogen, can be produced by steam reforming, partial oxidation, autothermal reforming and dry reforming from natural gas and further gaseous and liquid hydrocarbons. The process for producing the synthesis gas can be selected, inter alia, depending on the desired synthesis gas composition.

Steam reforming: CH₄+H₂O [symbol] CO+3H₂

Partial oxidation: CH₄+½O₂ [symbol] CO+2H₂

Autothermal reforming with O₂/CO₂: 2CH₄+O₂+CO₂→3H₂+3CO+H₂O

Autothermal reforming with O₂/H₂O: 4CH₄+O₂+2H₂O→10H₂+4CO

Dry reforming: CH₄+CO [symbol] 2CO+2H₂

Furthermore, the water-gas equilibrium must be taken into account.

(Water-gas shift reaction): CO+H₂O [symbol] CO₂+H₂

Dry reforming describes the reaction of hydrocarbons such as methane with CO₂ to give CO and hydrogen. The hydrogen formed in the reaction has a tendency to be depleted in reaction with the CO₂ by means of reverse water-gas shift reaction. Typical catalysts for dry reforming are noble metal catalysts such as nickel or nickel alloys.

Autothermal reforming uses oxygen and CO₂ or steam to convert the methane to synthesis gas. The methane is in part partially oxidized with oxygen. Autothermal reforming is a combination of partial oxidation and steam reforming. Autothermal is reforming combines the advantage of partial oxidation (provision of thermal energy) with the advantage of steam reforming (higher hydrogen yield), which optimizes efficiency.

In the production of higher alcohols from CO-containing synthesis gases, in addition to the preferred alcohols and possibly alkenes that alongside the alcohols are also considered to be products of value, there is formation of alkanes, such as for example methane and CO₂ as by-products.

In their dissertation on the topic “Design of a process for the production of the higher alcohols ethanol and propanol from synthesis gas”, Bastian Krause describes a process that is directed to the production of higher alcohols on the basis of synthesis gas produced from biomass. The CO₂ formed is removed in a complex CO₂ scrubbing operation, meaning that the CO₂ is no longer available for the production of chemical compounds, and the purified synthesis gas is then converted to alcohols. However, the CO₂ scrubbing, which is accompanied by a removal of the CO₂, lowers the carbon efficiency. The methane formed as by-product is (partially) converted to synthesis gas via a partial oxidation with oxygen.

Due to increased efforts in recent years to reduce greenhouse gas emissions, there has been increased interest in converting CO₂ and CO₂-containing gases, such as blast furnace gas, to chemical products. The direct conversion of CO₂ to higher alcohols generally affords a product mixture of CO, alcohols, methane and other oxygenates. CO is formed as main product with a selectivity of up to 85%, the CO₂ conversion is up to 30% (Advanced Materials Research Online, Vol. 772, pp 275-280; Acta Phys. —Chim. Sin. 2018, 34 (8), 858-872, Chemical Engineering Journal 240 (2014) 527-533; Catal Lett (2015) 145:620-630; Applied Catalysis A, General 543 (2017), 189-195). The formation of the higher alcohols is described as a sequence of reactions via the formation of CO by rWGS reaction and subsequent conversion of the CO to higher alcohols. The direct conversion of CO₂ generally leads to increased formation of by-products such as methane. The conversion of CO₂ to CO beforehand, e.g. by reverse water-gas shift reaction, is therefore advantageous.

The lower selectivity of the conversion of CO₂ to higher alcohols—which taken alone constitutes a considerable problem for carbon efficiency—has an additional disadvantageous effect on process efficiency and process economics at various points, since this manifests for example, in addition to in the accumulation of N₂ during the recycling of the unconverted synthesis gas, in the formation of methane as by-product—which is to be removed in a complex manner either by partial oxidation and conversion to CO/H₂ or by discharge—which results in an increase in the inert fraction in the synthesis gas. As a result, the plant apparatuses and process streams have to be dimensioned larger and the residual amount of synthesis gas increases as the inert fraction rises. While the separation of CO and N₂ by means of cryogenic separation methods is possible in principle, it is very cost-intensive.

Proceeding from the prior art described above, an object of the invention is accordingly that of providing a plant complex and a process for operating a plant complex which in an economical and particularly efficient manner make it possible to synthesize CO₂-containing gas, especially blast furnace gas and/or converter gas, to higher alcohols, especially ethanol, propanol and butanol, while in the process achieving maximal utilization of the carbon present in CO and CO₂ and simultaneously minimizing the required amount of H₂ that has to be externally provided.

This object is achieved according to the invention by a plant complex of the generic type mentioned at the outset, wherein the plant complex has a reformer being connected to the gas conducting system, in which reformer the CO₂-containing gas reacts with H₂ and/or hydrocarbons to give a CO— and H₂-containing synthesis gas mixture, the reformer is connected to a reactor for producing higher alcohols in which the synthesis gas mixture optionally reacts with further H₂ to give a gas/liquid mixture containing higher alcohols, and wherein the gas/liquid separation system for separating off the alcohols of the gas/liquid mixture is connected to the reactor for producing higher alcohols. The reformer may for example be a reformer for autothermal reforming or dry reforming.

This object is also achieved according to the invention by a process of the generic type mentioned at the outset, wherein the following process steps are performed:

V1) reacting hydrocarbons with the CO₂-containing gases and/or CO₂ and/or O₂ and/or H₂O as oxygen sources to give a CO— and H₂-containing synthesis gas mixture in the reformer,

V2) reacting the synthesis gas mixture with H₂ to give a gas/liquid mixture containing higher alcohols in the reactor for producing higher alcohols and

V3) separating off the alcohols of the gas/liquid mixture in the gas/liquid separation system from the gas components.

The plant complex according to the invention has a unit that produces CO₂-containing gases, for example a blast furnace for the production of pig iron and a converter steel works for the production of crude steel, and a gas conducting system for the CO₂-containing gases. An essential constituent of the plant complex according to the invention is that the plant complex has a reformer connected to the gas conducting system. In this reformer, the CO₂-containing gas reacts with H₂ and/or hydrocarbons to give a CO— and H₂-containing synthesis gas mixture which serves as a starting material for obtaining the higher alcohols.

The reaction of the synthesis gas mixture with H₂ to give higher alcohols within the plant complex according to the invention is then effected in one or more reactors for producing higher alcohols. In this/these reactors, the synthesis gas mixture is catalytically converted into a gas/liquid mixture containing higher alcohols. The CO₂ balance of the plant complex is therefore significantly improved, especially when using “green” H₂, which is for example produced by water electrolysis.

In addition, the plant complex according to the invention has a gas/liquid separation system in which the alcohols, in particular the higher alcohols, and possibly also the alkanes and alkenes of the gas/liquid mixture, are separated off. The alcohols obtained may then for example be marketed as a product mixture, especially as fuel additive, or be separated into the individual alcohols in a distillation process. The alkanes and alkenes can likewise be sent for industrial use, with the H₂ present in the alkanes preferably being recovered and the alkenes being sent for further value creation.

In a preferred development of the plant complex according to the invention, the unit that produces CO₂-containing gases comprises a blast furnace for the production of pig iron and a converter steel works for the production of crude steel, wherein the gas conducting system conducts the gases formed in the production of pig iron and/or the production of crude steel. In such an application scenario, the plant complex according to the invention can in an economically particularly efficient manner synthesize the CO₂-containing blast furnace gas and/or converter gas to higher alcohols and in the process achieve maximal utilization of the carbon present in CO and CO₂.

For the purposes of the present invention, higher alcohols are understood in particular to be ethanol, propanol and butanol.

In a development of this preferred plant complex, the unit that produces CO₂-containing gases furthermore comprises a coke-oven plant, wherein the gas conducting system includes a gas distribution for coke-oven gas that is formed in a coking process in the coke-oven plant. This can increase the self-sufficiency and the economic viability of the plant complex with regard to the H₂ needed, since H₂ is present to a large part in the coke-oven gas and after separating off secondary components can be made available for obtaining higher alcohols by the plant complex according to the invention.

Examples of CO₂-containing gases likewise considered for the plant complex according to the invention are flue gases, COREX or FINEX gases, and industrial process gases from lime kiln plants, cement plants, biogas plants, bioethanol plants and waste incineration plants.

According to a development of the plant complex according to the invention, the plant complex includes a gas compression unit for compressing the gases to the respective reaction pressure in the reformer and the reactor for producing higher alcohols.

In order to protect the catalyst disposed in the reactor for producing higher alcohols, according to a preferred development the plant complex of the invention includes a gas purification unit. As a result, the service life of the catalyst located in the reactor for producing higher alcohols can be increased, in that aggressive constituents of the CO₂-containing gases, in particular cyanides and sulfur or ammonium compounds, are removed.

In a preferred development, the plant complex according to the invention has a gas/liquid separation system for separating the gaseous and the liquid components of the product mixture of the alcohol reactor and for returning the gas components of the gas/liquid mixture has a gas recycle conduit connected to the reformer and/or to the reactor for producing higher alcohols. The recycling can be effected in the reformer, in order to convert possible by-products, in particular hydrocarbons present in the synthesis gas mixture and CO₂ to CO, or in the reactor for producing higher alcohols in order to increase the conversion of the synthesis gas. The choice of reaction regime, i.e. the proportion of recycling into the reformer and/or the reactor for producing higher alcohols is in this case dependent on the concentration of hydrocarbons and CO₂ in the gas phase, since the carbon efficiency can be optimized in a particularly advantageous manner by this.

A similar situation applies for the preferred variant of the invention, where the gas/liquid separation system has a recycle conduit connected to the reformer for returning, into the reformer, the liquid components of the gas/liquid mixture, in particular higher hydrocarbons, which are present in liquid phase in the gas/liquid mixture as by-products. The carbon efficiency can also be further improved by this.

Discharge of the synthesis residual gas allows an increase in concentration of inert components to be prevented in one development of the plant complex according to the invention. The result of this is that the plant size is advantageously kept compact, since an unnecessary entrainment of inert components in the gas is effectively prevented. This also reduces the plant costs and operating costs. An increase in concentration of inert components, in particular of N₂, can also be prevented by passing the gas components departing the gas/liquid separation system through a membrane for separating off nitrogen.

According to a development of this further-developed plant complex of the invention, connected to the outlet for the discharge of the synthesis residual gas is a pressure swing adsorption unit for the recovery of H₂ by pressure swing adsorption and subsequent recycling into the reformer and/or the reactor for producing higher alcohols. This increase in hydrogen yield can result in a reduction in the amount of externally produced hydrogen required, as a result of which the dependence on expensive external H₂ can be further decreased, with the result that the economic viability of the plant complex can be additionally raised.

In a preferred development, the reformer of the plant complex according to the invention is designed for operation in the temperature range of from 600 to 1200° C. This allows the equilibrium of the reverse water-gas shift reaction to be adjusted in a particularly advantageous manner, in particular to be shifted towards the product side. In the temperature range specified, a relatively high proportion of CO and H₂O is established as equilibrium state. It has been found that higher alcohols are obtained particularly efficiently in the reactor for producing higher alcohols as a result. A particularly high conversion of the CO₂ in smelter gases to CO in the reformer is achieved in the temperature range of from 1050 to 1150° C.

The process according to the invention is conducted in a plant complex comprising a unit that produces CO₂-containing gases, a gas conducting system for CO₂-containing gases, a reformer, a reactor for producing higher alcohols and a gas/liquid separation system.

In a first step of the process according to the invention, the hydrocarbons are reacted in the reformer with the CO₂-containing gases and/or CO₂ and/or O₂ and/or H₂O as oxygen sources to give a CO— and H₂-containing synthesis gas mixture.

For improving the carbon efficiency, it is preferable to react excess CO₂ with H₂ likewise to give CO. This may be effected in the reformer itself or in a further reactor. A second step of the process according to the invention comprises reacting the synthesis gas mixture, optionally with addition of H₂, to give a gas/liquid mixture containing higher alcohols in the reactor for producing higher alcohols. Preference is given to using a composition of the synthesis gas having an H₂:CO ratio of from 1:2 to 2:1.

Finally, in a third step of the process according to the invention, the alcohols of the gas/liquid mixture in the gas/liquid separation system are separated off from the gas components, so that the higher alcohols are produced in a carbon-efficient manner and for example can be separated into the different alcohols in a downstream distillation process. The alkanes and alkenes are also particularly preferably separated off.

The CO₂-containing gases are particularly preferably coke-oven gas and/or blast furnace gas and/or converter gas, since the process according to the invention has particular potential for improving the carbon efficiency in the production of coke, crude steel and pig iron.

According to a particularly preferred variant of the process according to the invention, the CO₂-containing gases are purified in a gas purification unit and/or compressed in a gas compression unit prior to the reaction with H₂ and/or hydrocarbons to give a CO— and H₂-containing synthesis gas mixture in the reformer. As a result of this, prior to the entry of the CO₂-containing gases into the reformer, firstly a minimum purity of the gas is ensured in order to protect the catalyst used in the production of the higher alcohols, and secondly the gas is brought to a defined—reaction rate influencing—pressure in order to be able to optimally perform the following process steps, in particular the synthesis of higher alcohols.

In the process according to the invention, the—preferably compressed and purified—gas is then passed into a reformer. In this reformer, the gas is reacted with H₂ and/or hydrocarbons to give a CO— and H₂-containing synthesis gas mixture, with CO₂ and/or O₂ and/or H₂O being used as oxygen sources. Using methane as an example, mention may be made of the following reactions taking place in the reformer, these proceeding depending on the concentrations of the respective components:

Dry reforming: CH₄+CO₂↔2CO+2H₂

Steam reforming: CH₄+H₂O↔CO+3H₂

Partial oxidation: CH₄+½O₂↔CO+2H₂

Reverse water-gas shift reaction: CO₂+H₂↔CO+H₂O

The synthesis gas produced by the reformer and for the production of the higher alcohols thus contains CO and CO₂ (residual content of unconverted CO₂). A particular feature is that the high reaction temperatures of the reforming make it possible to set the equilibrium of the reverse water-gas shift reaction, optionally also with addition of hydrogen and using a suitable catalyst for the reverse water-gas shift reaction, and in particular to shift it towards the product side. It has been found that this can significantly influence the efficiency of the conversion of the synthesis gas mixture to higher alcohols in the reactor for producing higher alcohols that is connected downstream of the reformer. Temperatures of >600° C. are required to shift the equilibrium of the water-gas shift reaction towards the product side. A particularly high conversion of the CO₂ in smelter gases to CO in the reformer or water-gas shift reactor is achieved when the reformer or water-gas shift reactor is operated in the temperature range of from 1050 to 1150° C.

In a development of the process according to the invention, the gas components are recycled into the reformer and/or the reactor for producing higher alcohols. According to the invention, the carbon efficiency of the conversion of the synthesis gas to higher alcohols can be increased by converting the by-products formed to alcohols in a further process step. The alkenes can for example be converted into alcohols by means of hydration. CO₂ can be hydrogenated to CO via the reverse water-gas shift reaction (rWGS). The alkanes can for example be converted into synthesis gas by steam reforming, partial oxidation, autothermal reforming and dry reforming, and recycled into the process. In particular, if “green” and possibly more expensive hydrogen produced using renewable energy is used in the production of the higher alcohols, then a conversion of the alkanes to synthesis gas is economically and environmentally advantageous with respect to the provision of hydrogen.

In the production of the higher alcohols according to the invention, the conversion of the alkanes formed as by-product and of the CO₂ formed as by-product and optionally of the CO₂ or CO₂-containing gases used as feed for the production of the synthesis gas can advantageously be combined via dry reforming or autothermal reforming, optionally also with addition of oxygen and/or water, in a reactor for synthesis gas production. The CO₂ in this case serves as an oxygen source for the reforming of the alkanes. When using CO₂ as feed for the synthesis gas production, the CO₂ is generally in excess with respect to the alkanes formed as by-products in the process for producing the higher alcohols. The aim is thus to convert the excess CO₂, optionally with addition of additional hydrogen, to CO by means of reverse water-gas shift reaction. The conversion of the CO₂ to CO and the shift of the equilibrium of the water-gas shift reaction can preferably be effected in the reactor for synthesis gas production (dry reforming or autothermal reforming) or else in a downstream reactor. Alternatively, the CO₂ or CO₂-containing gas used as feed for the production of the synthesis gas can be fed partially or completely directly into the reactor for the water-gas shift reaction.

The (thermal) energy required for the reforming (e.g. dry reforming) and the reverse water-gas shift reaction can be provided in the plant complex according to the invention, made up of blast furnace, coking plant and plant for producing the higher alcohols, for example by the combustion of the blast furnace gas, of the coke-oven gas, of the offgas from the coke-oven gas PSA or from off gases from the chemical plant. When producing hydrogen by means of electrolysis, the oxygen formed as co-product can be used for the partial oxidation or autothermal reforming of the hydrocarbons.

According to a preferred variant of the process according to the invention, the H₂ present in the synthesis residual gas is recovered by pressure swing adsorption in a pressure swing adsorption unit and is supplied to the reformer and/or to the reactor for producing higher alcohols, with the result of increasing the hydrogen yield, reducing the dependence on external H₂ sources such as for example from an expensive water electrolysis, and increasing the economic viability.

The same advantage is achieved in a preferred development of the process according to the invention when H₂ is obtained from compressed coke-oven gas by pressure swing adsorption in a pressure swing adsorption unit and is supplied to the reformer and/or to the reactor for producing higher alcohols.

In a preferred development of the process according to the invention, due to the possible conversion of methane and other hydrocarbons in the reformer, the alkanes, such as methane, ethane, propane and butane, formed as by-products in the process for producing higher alcohols can advantageously be converted back to synthesis gas in the reformer and recycled into the process. Optionally, methanol and/or the alkenes can also be converted back into synthesis gas in the reformer. The alkenes can also be synthesized to higher alcohols, in order to maximize the production of higher alcohols.

Particular preference within the scope of a development of the process according to the invention is given to operating the reformer in a temperature range from 600 to 1200° C. As a result, the process according to the invention exploits the knowledge that the efficiency of the synthesis of the higher alcohols is influenced by the CO concentration in the reverse water-gas shift reaction being influenced by the choice of the temperature range. In the ideal case, the reaction conditions are selected such that a high CO₂ conversion is achieved and only little, if any, methane and/or alkanes are formed or remain in the gas mixture.

Advantageous developments become apparent from the dependent claims, the following description and the figures.

The invention is described below on the basis of exemplary embodiments with reference to the enclosed drawings. In the figures:

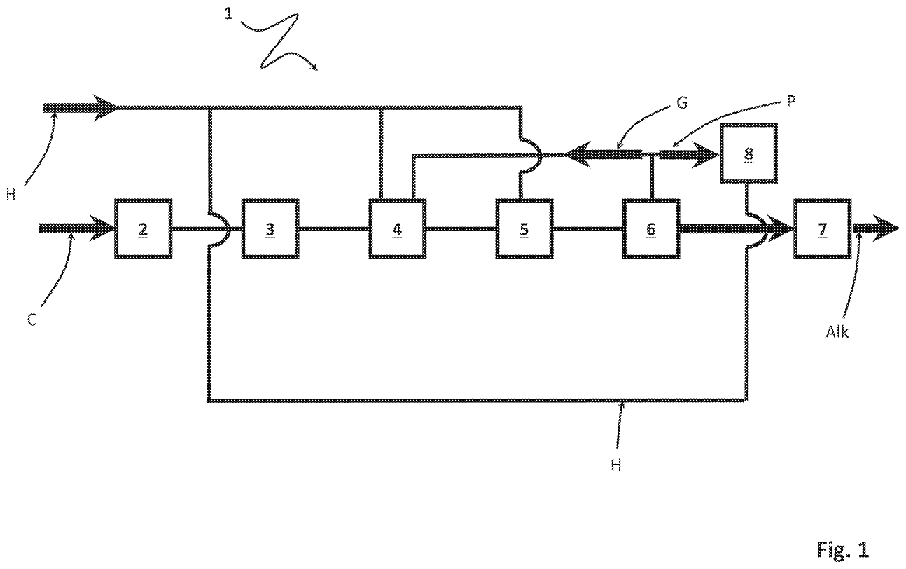

FIG. 1: A schematic diagram of a plant complex according to the invention,

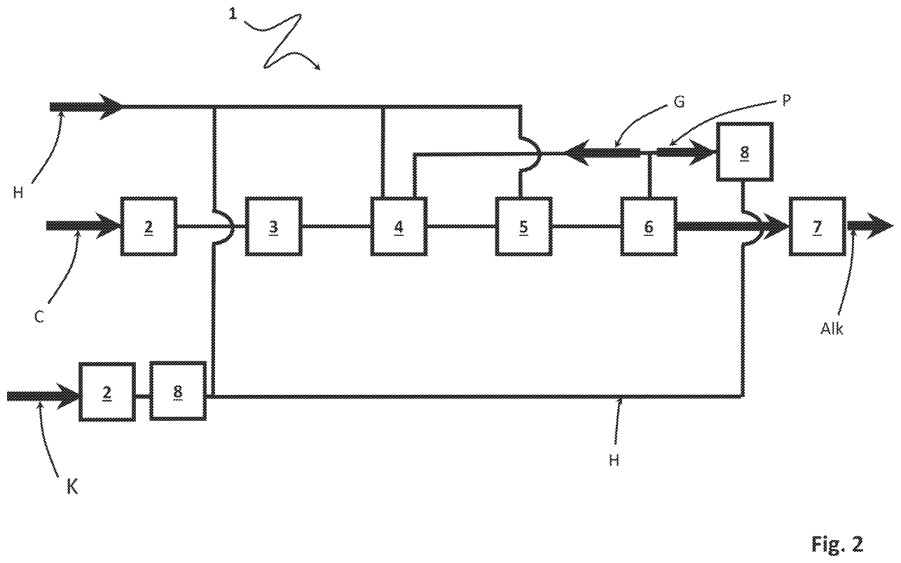

FIG. 2: A schematic diagram of a further plant complex according to the invention,

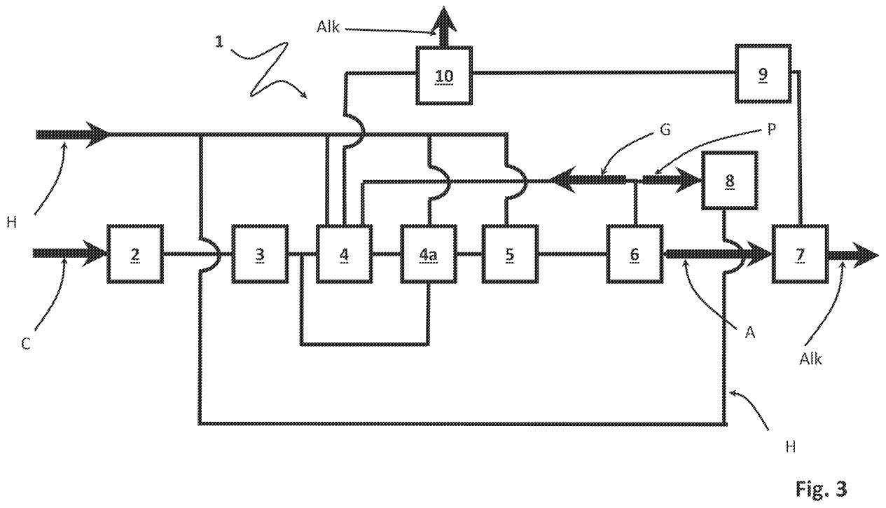

FIG. 3: A schematic diagram of a further plant complex according to the invention, and

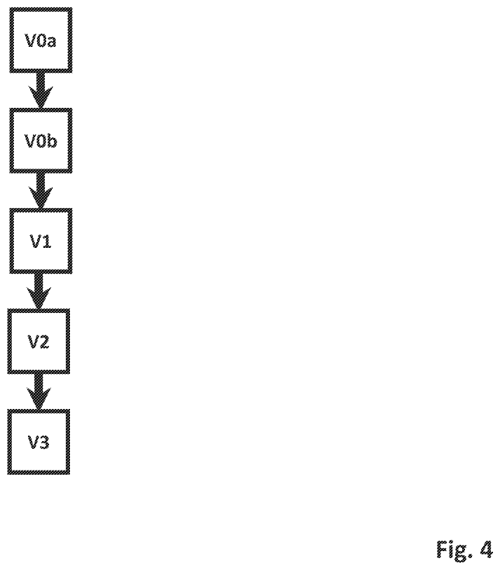

FIG. 4: A schematic diagram of the process according to the invention.

In the various figures, identical parts are always provided with the same reference signs and are therefore also generally each named or mentioned only once.

FIG. 1 shows an example of a plant complex 1 according to the invention, in which CO₂-containing gases C from a unit that produces CO₂-containing gases are brought in a gas compression unit 2 to a pressure that is predeterminable for the following processes, in order thereby to be able to adjust the reaction rate for the following chemical reactions. Then, in a gas purification unit 3, the compressed CO₂-containing gases are purified of chemical substances that impair the catalyst of the reactor for producing higher alcohols in terms of its functioning and service life, in particular cyanides and sulfur and ammonium compounds.

Hydrocarbons then react with the CO₂-containing gases C and/or CO₂ and/or O₂ and/or H₂O as oxygen sources to give a CO— and H₂-containing synthesis gas mixture in a reformer 4. The synthesis gas produced by the reformer 4 and for the production of the higher alcohols contains CO and CO₂. It is a particular advantage that, when using the reformer 4, it can be used to adjust the equilibrium of the reverse water-gas shift reaction. Optimally, this is shifted to the product side, so that a particularly high conversion of the CO₂, for example from smelter gases, to CO is achieved, which in turn improves the efficiency of the synthesis of higher alcohols. The adjustment of the equilibrium of the reverse water-gas shift reaction, so as to achieve a particularly high conversion of the CO₂ to CO, is achieved in a particularly advantageous manner in the plant complex 1 according to the invention by operating the reformer 4 in a temperature range from 600 to 1200° C., in particular 1050 to 1150° C.

After the synthesis gas mixture has been produced in the reformer 4 with the highest possible content of CO, it is catalytically reacted, in a reactor for producing higher alcohols 5, with H₂ to give a gas mixture containing higher alcohols, whereupon this gas mixture is separated into a liquid phase and a gas phase.

Subsequently—as is also shown in FIG. 1—the gas/liquid mixture for separating off the alcohols is passed into a gas/liquid separation system 6 which is connected to the reactor 5 and in which the higher alcohols, in particular ethanol, propanol and butanol, are separated off and in a downstream distillation unit 7 are separated into their individual constituents.

The gas/liquid separation system 6 has a gas recycle conduit connected to the reformer for returning the gas components of the gas/liquid mixture, in order to recycle the gas components G to further improve the carbon efficiency.

In the plant complex illustrated in FIG. 1, the H₂ for the reformer 4 and the reactor for producing higher alcohols 5 is provided, inter alia, via H₂ recovery in a pressure swing adsorption unit 8 from the synthesis residual gas P departing the reformer 4, which is separated off from the gas components G, in order to reduce the dependence on external H₂ sources and increase the H₂ self-sufficiency.

Particular preference with regard to minimizing the dependence on external H₂ sources is given, in accordance with the further-developed plant complex according to the invention illustrated in FIG. 2, to providing the H₂ for the reformer 4 and the reactor for producing higher alcohols 5 via the purification/obtaining of the H₂ from coke-oven gas (H₂-rich) K by means of H₂ recovery (pressure swing adsorption) and also the recovery of H₂ from the synthesis residual gas P.

FIG. 3 shows a further preferred configuration of the plant complex according to the invention. In this plant complex, connected upstream of the reactor for producing higher alcohols 5 is an additional reactor for optimizing/fine-tuning the synthesis gas composition 4a, in which in particular the equilibrium of the water-gas shift reaction can be adjusted, as a result of which the efficiency when producing higher alcohols can be further improved. In addition, this plant complex according to the invention has a further stage by means of which separation of the alcohols from the hydrocarbons is made possible, for example a distillation unit 7. The hydrocarbons separated off are supplied to a hydration unit 9 in which the alkenes are converted to alcohols. The alcohols obtained by the hydration are then separated off from the alkanes and unconverted alkenes to be recycled into the process in an alcohol/alkane separation device 10. The alkanes and alkenes are preferably recycled by introduction into the reformer.

FIG. 4 shows a schematic diagram of the process according to the invention. In process step V0a, for protecting the catalyst disposed in the reactor for producing higher alcohols, aggressive constituents of the CO₂-containing gases, in particular cyanides and sulfur or ammonium compounds, are removed in the gas purification unit in order to increase the service life of the catalyst located in the reactor for producing higher alcohols. Subsequently, the CO₂-containing gases are brought to a defined pressure V0b in a gas compression unit in order to be able to perform the following process steps optimally. A multiplicity of different compressors may also be provided, since the gas purification and the gas synthesis proceed at different pressures. The CO₂-containing gases are then reacted in the reformer 4 with H₂ and/or hydrocarbons to give a CO— and H₂-containing synthesis gas mixture, which is then passed into the reactor for producing higher alcohols 5. Finally, the alcohols A of the gas/liquid mixture in the gas/liquid separation system 6 are separated off from the gas components.

### LIST OF REFERENCE SIGNS

1 Plant complex
2 Gas compression unit
3 Gas purification unit
4 Reformer
4a Reactor for adjusting the synthesis gas composition
5 Reactor for producing higher alcohols
6 Gas/liquid separation system
7 Distillation unit
8 Pressure swing adsorption unit
9 Hydration unit
10 Alcohol/alkane separation device
A Alcohols, alkanes, alkenes
Alk Alcohols
C CO₂-containing gases
G Gas components
H H₂
K Coke-oven gas
P Synthesis residual gas
V0a Gas purification
V0b Gas compression
V1 Reaction of the CO₂-containing gases to give synthesis gas mixture
V2 Reaction of the synthesis gas mixture to give a gas/liquid mixture containing higher alcohols
V3 Separating-off of the liquid higher alcohols

The invention claimed is:

1.-15. (canceled)

16. A plant complex comprising:
a unit that produces CO₂-containing gases;
a gas conducting system for CO₂-containing gases;
a gas/liquid separation system; and
a reformer that is connected to the gas conducting system, wherein the reformer is configured so that the CO₂-containing gases react with H₂ and/or hydrocarbons in the reformer to give a CO— and H₂-containing synthesis gas mixture, the reformer being connected to a reactor for producing higher alcohols in which the synthesis gas mixture reacts with H₂ to give a gas/liquid mixture containing higher alcohols,
wherein for separating off the alcohols of the gas/liquid mixture, the gas/liquid separation system is connected to the reactor for producing higher alcohols.

17. The plant complex of claim 16 wherein the unit comprises a blast furnace configured to produce pig iron and a converter steel works configured to produce crude steel, wherein the gas conducting system conducts gases formed in the production of pig iron and/or gases formed in the production of crude steel.

18. The plant complex of claim 17 wherein the unit comprises a coke-oven plant, wherein the gas conducting system includes a gas distribution for coke-oven gas that is formed in a coking process in the coke-oven plant.

19. The plant complex of claim of claim 16 comprising a gas compression unit.

20. The plant complex of claim 16 comprising a gas purification unit.

21. The plant complex of claim 16 wherein the gas/liquid separation system is configured to separate off alkanes and alkenes of the gas/liquid mixture.

22. The plant complex of claim 16 wherein the gas/liquid separation system includes a recycle conduit connected to the reformer that is configured to return, into the reformer, gas components including CO, CO₂, H₂, and methane that are present in the gas/liquid mixture as reactants and by-products.

23. The plant complex of claim 22 comprising a pressure swing adsorption unit that is connected to an outlet for discharge of synthesis residual gas, wherein the pressure swing adsorption unit is configured to recover H₂ by pressure swing adsorption and then recycle the H₂ into the reformer and/or the reactor for producing higher alcohols.

24. The plant complex of claim 16 comprising:
a second unit that is downstream of the gas/liquid separation system and is configured to separate the alcohols from the hydrocarbons;
a hydration unit that is connected to the second unit and is configured to convert alkenes to alcohols; and
an alcohol/alkane separation device, wherein the hydration unit is connected to the alcohol/alkane separation device, wherein the hydration unit and the alcohol/alkane separation device are configured such that the alcohols obtained by hydration are separated off from the alkanes and unconverted alkenes to be recycled into the plant.

25. A process for producing higher alcohols from CO₂-containing gases with a plant complex comprising a unit that produces CO₂-containing gases, a gas conducting system for CO₂-containing gases, a reformer, a reactor for producing higher alcohols, and a gas/liquid separation system, wherein the process comprises:
V1) reacting hydrocarbons with the CO₂-containing gases and/or CO₂ and/or O₂ and/or H₂O as oxygen sources to give a CO— and H₂-containing synthesis gas mixture in the reformer;
V2) reacting the synthesis gas mixture with H₂ to give a gas/liquid mixture containing higher alcohols in the reactor for producing higher alcohols; and
V3) separating off liquid alcohols of the gas/liquid mixture in the gas/liquid separation system from gas components.

26. The process of claim 25 comprising:
recovering the H₂ present in the synthesis residual gas by pressure swing adsorption in a pressure swing adsorption unit; and
supplying the H₂ to the reformer and/or to the reactor for producing higher alcohols.

27. The process of claim 25 comprising:
obtaining H₂ from compressed coke-oven gas by pressure swing adsorption in a pressure swing adsorption unit; and
supplying the H₂ to the reformer and/or to the reactor for producing higher alcohols.

28. The process of claim 25 comprising operating the reformer in a temperature range from 600° C. to 1200° C.

29. The process of claim 25 comprising separating alkanes and alkenes of the gas/liquid mixture off in the gas/liquid separation system.

30. The process of claim 25 wherein after separating off liquid alcohols of the gas/liquid mixture in the gas/liquid separation system from gas components, the process comprises separating the alcohols from the hydrocarbons and supplying the hydrocarbons to a hydration unit where alkenes are converted to alcohols, wherein in an alcohol/alkane separation device the alcohols obtained by hydration are then separated off from alkanes and unconverted alkenes to be recycled into the process.
